(19) United States
(12) Patent Application Publication
(10) Pub. No.: US 2024/0306965 A1
(43) Pub. Date: Sep. 19, 2024

(54) Line Draw Device for Direct Arterial Blood Sampling

(71) Applicant: Becton, Dickinson and Company, Franklin Lakes, NJ (US)

(72) Inventors: Jonathan Karl Burkholz, Salt Lake City, UT (US); Yiping Ma, Layton, UT (US)

(21) Appl. No.: 18/603,324

(22) Filed: Mar. 13, 2024

Related U.S. Application Data
(60) Provisional application No. 63/452,025, filed on Mar. 14, 2023.

(51) Int. Cl. A61B 5/15

(52) U.S. Cl. CPC A61B 5/150992; A61B 5/15003; A61B 5/150061; A61B 5/15019; A61B 5/150213; A61B 5/150236; A61B 5/150244

(57) ABSTRACT

A blood draw device for performing arterial blood collection includes a catheter tube, a housing configured to movably receive the catheter tube, and an advancement member movably coupled to the housing to move the catheter tube between first and second positions, with a distal end of the catheter tube disposed beyond a distal end of the housing and past a distal tip of the indwelling arterial catheter when in the second position. A secondary catheter is coupled to the advancement member and extends out proximally therefrom, with a collection device coupled to the secondary catheter so as to be in fluid communication with the catheter tube. The blood draw device is configured such that a fluid path formed by the catheter tube and secondary catheter has a controlled geometric factor, defined as a ratio of a length of the fluid path to an inner diameter of the fluid path.

### CROSS-REFERENCE TO RELATED APPLICATION

The present application claims priority to U.S. Provisional Application No. 63/452,025 entitled “Line Draw Device for Direct Arterial Blood Sampling” filed Mar. 24, 2023, the entire disclosure of which is hereby incorporated by reference.

### BACKGROUND OF THE INVENTION

### Field of the Invention

The present invention is directed to a blood draw device for direct arterial blood collection utilizing an indwelling arterial catheter.

### BACKGROUND OF THE INVENTION

Arterial catheterization is a vital procedure that is used ubiquitously in the hospital setting, both in critically injured and perioperative patients. Arterial catheters are used to continuously monitor and measure blood pressure, heart rate, and pulse contour to allow for immediate recognition of aberrant hemodynamic events and initiation of appropriate treatment. Arterial catheters are also used to provide samples for blood gas analysis without the morbidity associated with repeat arterial puncture. In use, an arterial catheter is usually inserted into the radial artery in the wrist, but can also be inserted into the brachial artery at the elbow, into the femoral artery in the groin, into the dorsalis pedis artery in the foot, or into the ulnar artery in the wrist.

It is recognized that the use of current arterial catheters and associated arterial blood gas and blood sampling systems can be complicated, have numerous workflow procedure steps that can be simplified, and can result in difficult to flush IV lines and an interruption in hemodynamic monitoring. For example, current systems used with arterial catheters require pulling a large clearing volume of blood out of the patient and into a large, complex fluid extension set prior to sampling, with the system then sampling from the extension set, pushing the large volume of blood back into the patient, and then flushing the large fluid path volume to clear the blood out of the extension set.

Accordingly, a need exists for an arterial access system and device for facilitating improved arterial line blood collection for arterial blood gas sampling and continuous pressure monitoring that overcomes the aforementioned limitations of existing systems and devices. The system and device would enable the acquisition of a blood sample from within the arterial of the patient, while eliminating the complexity and complication that come with the current systems and approaches.

### SUMMARY OF THE INVENTION

Provided herein is a blood draw device useable with an indwelling arterial catheter for performing arterial blood collection. The blood draw device includes a catheter tube, a housing having a proximal end portion and a distal end portion and defining an inner volume configured to movably receive the catheter tube, and an advancement member movably coupled to the housing and configured to move relative to the housing to move the catheter tube between a first position, in which the catheter tube is disposed within the housing, and a second position, in which a distal end of the catheter tube is disposed beyond the distal end portion of the housing and past a distal tip of the indwelling arterial catheter. The blood draw device also includes a secondary catheter coupled to the advancement member and extending out proximally therefrom and out through the proximal end portion of the housing, the secondary catheter in fluid communication with the catheter tube, and a collection device coupled to the proximal end of the secondary catheter, so as to be in fluid communication with the secondary catheter and the catheter tube. A fluid path formed by the catheter tube and the secondary catheter has a controlled geometric factor, Gf, defined as a ratio of a length of the fluid path, L, to an inner diameter of the fluid path, D.

In some embodiments, the blood draw device includes a coupler connected to a proximal end of the secondary catheter, and wherein the collection device is connected to the coupler.

In some embodiments, the collection device is a vacuum-assisted collection device.

In some embodiments, the collection device is a non-vacuum-assisted collection device.

In some embodiments, wherein the coupler includes a venting feature configured to selectively vent the non-vacuum-assisted collection device.

In some embodiments, the fluid path has a geometric factor, Gf, equal to or greater than 35/Dₘᵢₙ³ for a non-vacuum-assisted collection device and between equal to or greater than 220/Dₘᵢₙ³ for a vacuum-assisted collection device.

In some embodiments, for the secondary catheter having a constant inner diameter, the geometric factor, Gf, is defined as:

$$\\ Gf = \frac{L}{{D}^{{}_{} 4}} .$$

In some embodiments, for the secondary catheter having have multiple sections with lengths L1, L2, L3 and inner diameters of D1, D2, D3, the geometric factor, Gf, is defined as:

$$\\ {G}_{f} = \frac{L 1}{D {1}^{{}_{} 4}} + \frac{L 2}{D {2}^{{}_{} 4}} + \frac{L 3}{D {3}^{{}_{} 4}} .$$

In some embodiments, for the secondary catheter having a changing inside diameter over the length thereof, the geometric factor, Gf, is defined as:

$${G}_{f} = {\int }_{0}^{L} \frac{d l}{{D ( l )}^{4}} .$$

In some embodiments, the housing and/or the advancement member are configured such that, when the catheter tube is moved to the second position, the distal end of the catheter tube extends out 10 mm or less past the distal tip of the indwelling arterial catheter.

In some embodiments, the housing and/or the advancement member are configured such that, when the catheter tube is moved to the second position, the distal end of the catheter tube extends out between 3-10 mm past the distal tip of the indwelling arterial catheter.

In some embodiments, the housing has a length of 103-110 mm or 122-129 mm.

In some embodiments, comprising a spacer positioned within the inner volume of the housing and proximally from the advancement member, the spacer limiting movement of the advancement member along the housing.

Also provided is a method of manufacturing an arterial blood draw device operable to collect an arterial blood sample. The method includes providing a catheter tube, housing the catheter tube within an inner volume of a housing having a proximal end portion and a distal end portion such the catheter tube is movable within the housing, and coupling an advancement member to the catheter tube and to housing, with the advancement member movable relative to the housing, so as to cause a corresponding movement of the catheter tube between a first position, in which the catheter tube is disposed within the housing, and a second position, in which a distal end of the catheter tube is disposed beyond the distal end portion of the housing. The method also includes connecting a secondary catheter to the advancement member to extend proximally from the advancement member and out through the proximal end portion of the housing, the secondary catheter in fluid communication with the catheter tube, and connecting a collection device at a proximal end of the secondary catheter, the collection device comprising one of a vacuum-assisted collection device and a non-vacuum-assisted collection device. The method further includes configuring the catheter tube and the secondary catheter to collectively provide a fluid path having a geometric factor, Gf, defined as a ratio of a length of the fluid path, L, to an inner diameter of the fluid path, D, with the geometric factor, Gf, having a first value or value range when the collection device is a vacuum-assisted collection device and having a second value or value range when the collection device is a non-vacuum-assisted collection device.

In some embodiments, the geometric factor, Gf, equal to or greater than 35/Dₘᵢₙ³ for a non-vacuum-assisted collection device and between equal to or greater than 220/Dₘᵢₙ³ for a vacuum-assisted collection device.

In some embodiments, the housing and/or the advancement member are configured such that, when the catheter tube is moved to the second position, the distal end of the catheter tube extends out 10 mm or less past the distal tip of the indwelling arterial catheter.

In some embodiments, for the secondary catheter having a constant inner diameter, the geometric factor, Gf, is defined as:

$$G f = \frac{L}{{D}^{4}} .$$

In some embodiments, for the secondary catheter having have multiple sections with lengths L1, L2, L3 and inner diameters of D1, D2, D3, the geometric factor, Gf, is defined as:

$${G}_{f} = \frac{L 1}{D {1}^{4}} + \frac{L 2}{D {2}^{4}} + \frac{L 3}{D {3}^{4}} .$$

In some embodiments, for the secondary catheter having a changing inside diameter over the length thereof, the geometric factor, Gf, is defined as:

$${G}_{f} = {\int }_{0}^{L} \frac{d l}{{D ( l )}^{4}} .$$

In some embodiments, the method of manufacturing the arterial blood draw device includes constructing the housing to have a length of 103-110 mm or 122-129 mm.

In some embodiments, constructing the housing and/or coupling the advancement member to the housing is performed such that, when the catheter tube is moved to the second position, the distal end of the catheter tube extends out 10 mm or less past the distal end portion of the housing.

### BRIEF DESCRIPTION OF THE DRAWINGS

FIG. 1 is a perspective view of an arterial access system, with a syringe connected to a blood draw device thereof, in accordance with an aspect of the disclosure;

FIG. 2 is a perspective view of a blood draw device useable with the arterial access system of FIG. 1, in accordance with an aspect of the disclosure;

FIG. 3 is an exploded view of the blood draw of FIG. 2;

FIG. 4 is a side view of the blood draw device of FIG. 2, showing the catheter tube in a first, retracted position;

FIG. 5 is a side view of the blood draw device of FIG. 2, showing the catheter tube in a second, extended position;

FIG. 6 is a perspective view of an arterial access system, with a vacutainer connected to the blood draw device thereof, in accordance with an aspect of the disclosure;

FIG. 7 is a perspective view of an arterial access system, with a diagnostic cartridge connected to the blood draw device thereof, in accordance with an aspect of the disclosure; and

FIG. 8 is a perspective view of an arterial access system, in accordance with an aspect of the disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

As used in this specification, the words “proximal” and “distal” refer to the direction closer to and away from, respectively. a user who would place the device into contact with a patient. Thus, for example, the end of a device first touching the body of the patient would be the distal end, while the opposite end of the device (e.g., the end of the device being manipulated by the user) would be the proximal end of the device.

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

The present invention is directed to an arterial access system having a blood draw device (alternatively, “line draw device”), along with a method of using the arterial blood draw device to advance a blood draw catheter tube into an indwelling arterial catheter and subsequently draw a blood sample from a patient.

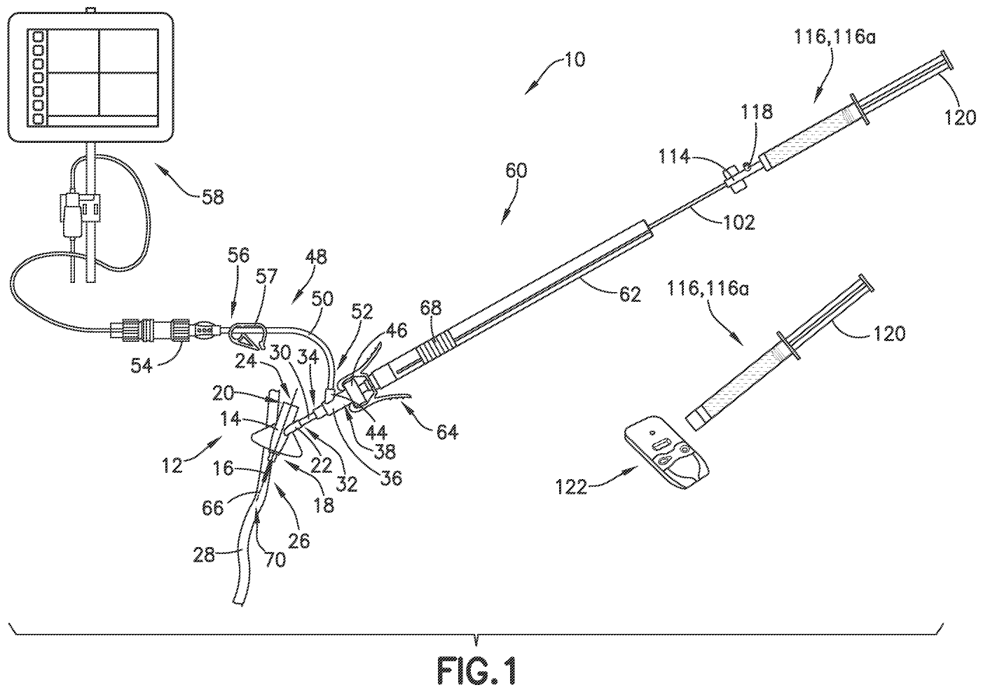

Referring to FIG. 1, shown is a non-limiting embodiment of an arterial access system 10 for facilitating improved arterial line blood collection for arterial blood gas sampling and continuous pressure monitoring. Arterial access system 10 may include a catheter assembly 12 having a catheter adapter 14 and associated catheter 16. The catheter adapter 14 may include a distal end 18 and a proximal end 20. In some embodiments, the catheter adapter 14 may include an additional adapter port 22 that may disposed between the distal end 18 and the proximal end 20 or disposed at the proximal end 20. The catheter adapter 14 may include a first lumen 24 extending through the distal end 18 and the proximal end 20, and the first lumen 24 may be sealed at proximal end 20 of catheter adapter 14. The catheter 16 extends from the distal end 18 of catheter adapter 14 and may be configured as an arterial catheter that is placed into an artery of the patient, with a distal end or tip 26 of the catheter 16 positioned appropriately within an artery 28 to enable a blood draw from the patient. In some embodiments, the catheter 16 may be inserted into an artery such that the distal tip 26 (an opening therein) faces upstream and into the arterial blood flow. Catheter 16 may be formed of any suitable material and may be of any useful length, as known to those of skill in the art.

In some non-limiting embodiments or aspects, the catheter assembly 12 may include a first fluid conduit 30 extending from the port 22. First fluid conduit 30 may be formed of any suitable material known to those of skill in the art and may have a distal end 32 and a proximal end 34. The distal end 32 of first fluid conduit 30 is coupled to port 22, while the proximal end 34 of first fluid conduit 30 may be coupled to a connector 36. Connector 36 may be a t-connector (e.g., one side port arranged at a 90 degree angle relative to a longitudinal axis of connector 36), a y-connector (e.g., one side port arranged at a 25, a 60, or a 75 degree angle relative to a longitudinal axis of connector 36), or any other type of connector known in the art. The connector 36 includes a second lumen 38 therethrough, having any number of branches suitable for the type of connector, such as a branch extending between distal and proximal ends 40, 42 of connector 36 and a branch provided to a port 44 of the connector 36.

In some non-limiting embodiments or aspects, catheter assembly 12 may include a needleless access connector 46 coupled to the proximal end 42 of connector 36, with the needleless access connector 46 providing an access port to the catheter assembly 12. The needleless access connector 46 may be configured as a split-septum connector or self-healing septum connector, as examples. In the illustrated embodiment, the access port provided by needleless access connector 46 is a near-patient access port close to the insertion site of the catheter 16, but it is recognized that an access port could be provided at other alternative locations close enough to the insertion site that allow for advancement of a blood draw catheter tube into the indwelling arterial catheter 16 and out beyond the distal tip thereof. For example, an access port that provides for advancement of a blood draw catheter tube into the indwelling arterial catheter 16 could be located on another connector, such as a proximal connector on an extension set (as described below) of the catheter assembly 12.

In some non-limiting embodiments or aspects, catheter assembly 12 may also include an extension set 48 coupled to the port 44 of the connector 36. The extension set 48 may include a second fluid conduit 50 coupled to port 44 at end 52 of the conduit 50 and a luer connection 54 at opposing end 56, with a clamp 57 provided on second fluid conduit 50 that allows for occlusion thereof. The extension set 48 may be used to provide a fluid path from the catheter assembly 12 to a hemodynamic monitoring device 58 that monitors blood pressure, heart rate, and/or pulse contour of a patient, based on arterial blood drawn through the catheter assembly 12. While the non-limiting embodiments of FIG. 1 shows a needleless access connector 46 arranged at connector 36, those of skill in the art will appreciate that a suitable needleless access connector may also be arranged at luer 54 of extension set 48.

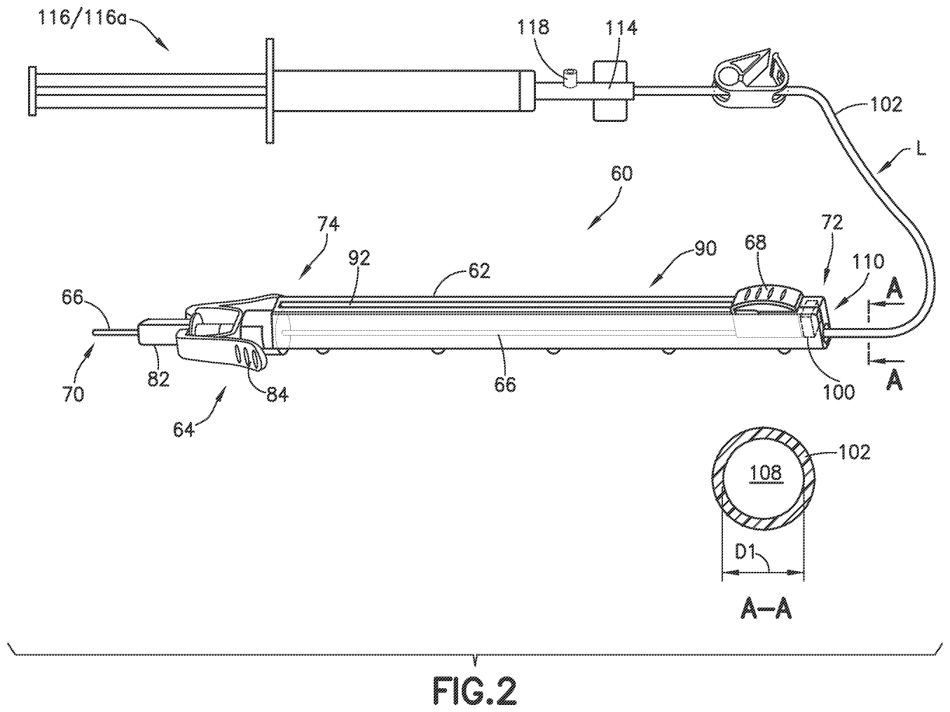

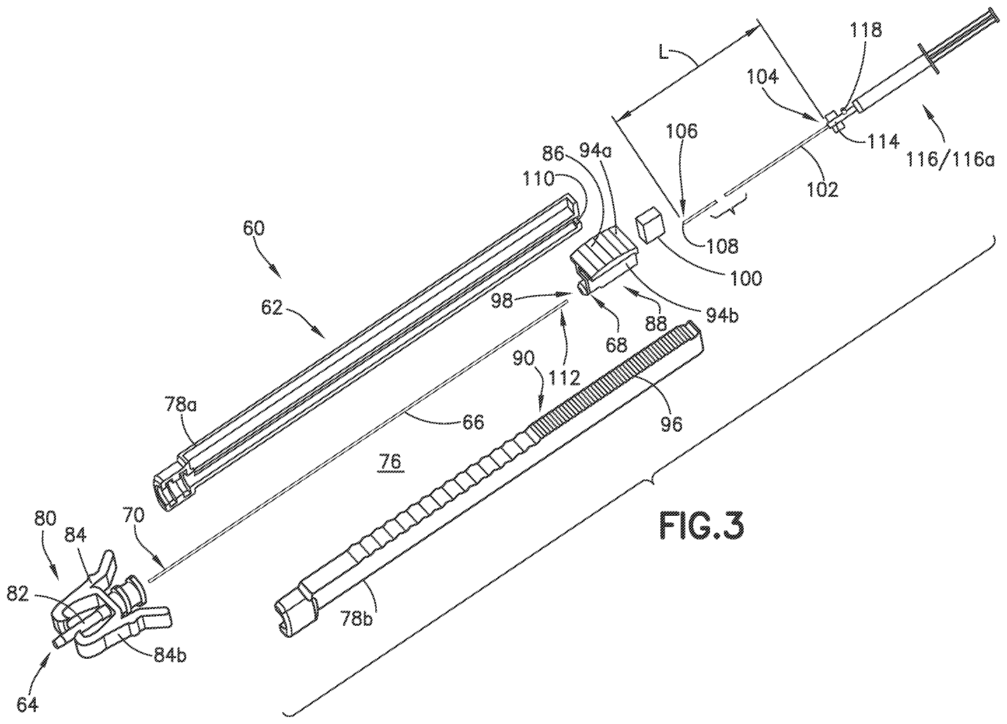

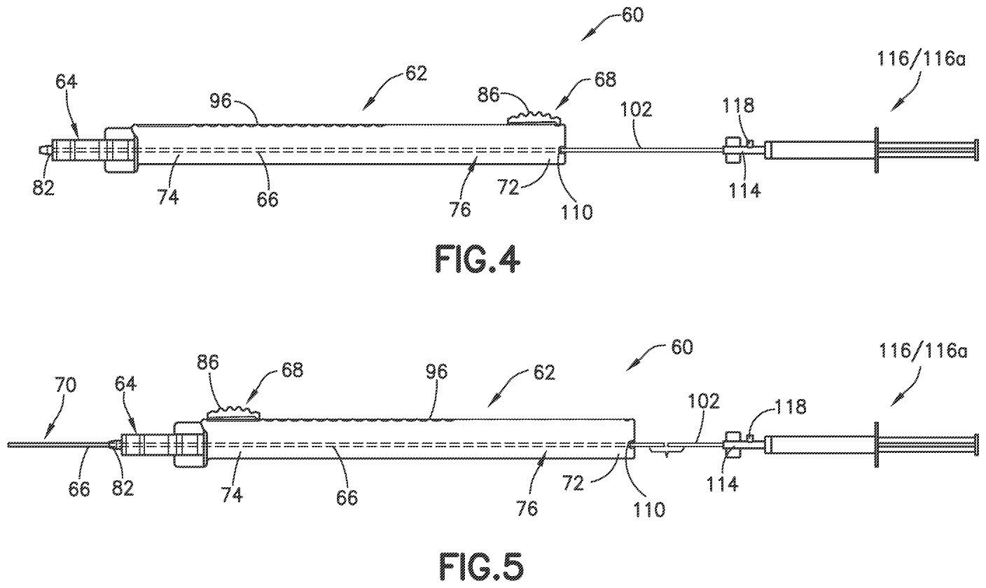

Arterial access system 10 further includes a blood draw device 60 (alternatively, “line draw device”) that may be operated to obtain a blood sample from the patient, with such a blood sample enabling blood gas analysis for example. As shown in FIG. 1 and in further detail in FIGS. 2-5, according to a non-limiting embodiment, the blood draw device 60 includes at least a housing 62, a coupling device 64, a catheter tube 66, and an advancement member 68. As will be described in further detail below, the catheter tube 66 is moveable within the housing 62 so as to provide for advancement of a portion of the catheter tube 66 from a first or retracted position inside the housing 62 (FIG. 3) to a second or advanced position outside of the housing 62 (FIG. 4), such that a distal end thereof may be routed into the catheter assembly 12. Once a portion of the catheter tube 66 has been routed into the catheter assembly 12 and out past the distal tip 26 of indwelling catheter 16, the catheter tube 66 may enable collection of a blood sample.

According to embodiments, the catheter tube 66 is sized to enable introduction thereof into the fluid path (i.e., into a lumen of catheter 16, lumen 24 of catheter adapter 14, and first fluid conduit 30) of catheter assembly 12 and for advancement therethrough. Accordingly, the catheter tube 66 can have an outer diameter (e.g., between a 10-gauge and a 30-gauge) that is smaller than the smallest lumen of the catheter assembly fluid path. The catheter tube 66 can have a length that is sufficient to place a distal end 70 of the catheter tube 66 in a desired position within the fluid path of the arterial access system 10. Thus, in one embodiment, the catheter tube 66 may have a length sufficient to provide for advancement of the distal end 70 thereof out from the housing 62 and through the catheter assembly (i.e., through connector 36, fluid conduit 30, catheter adapter 14 and catheter 16), and all the way out past the distal tip 26 of catheter 16.

In some embodiments, and in order to accommodate positioning of the catheter tube 66 within catheter 16 and advancement thereof out past the distal tip 26 of catheter 16, the distal end 26 of the catheter 16 may include fenestrations formed therein. The fenestrations at the distal end 26 of the catheter 16 serve to maintain fluid continuity from the artery and through the catheter 16 to the hemodynamic monitoring device 58 connected to the proximal end of extension set 48—i.e., the fenestrations prevent catheter tube 66 from occluding the catheter 16 at the distal tip 26 thereof and cutting off accurate pressure monitoring. The fenestrations in catheter 16 thus provide the ability to continue monitoring arterial pressure during a sampling procedure performed by blood draw device 60 as catheter tube 66 is advanced.

In accordance with aspects of the disclosure, it is recognized that the use of the blood draw device 60 with the indwelling arterial catheter 16 may differ from use of a blood draw device with an indwelling peripheral IV catheter. That is, unlike peripheral IV catheters that are used in the vein, arterial catheters have the distal end and opening facing upstream and into the oncoming arterial blood flow. The arterial blood flow is also typically at a higher flow rate and pressure and therefore, some of the challenges and complications seen with peripheral IV catheters do not appear with arterial catheters, such as thrombus development downstream from the catheter tip in the venous side, making aspiration and line draw difficult through the dwell period of a PIV. Accordingly, use of the blood draw device 60 with indwelling arterial catheter 16 includes different requirements, optimization targets and design considerations. For example, in accordance with some non-limiting embodiments of the disclosure, the blood draw device 60 may be configured to extend the distal end 70 of the catheter tube 66 out beyond the distal tip 26 of the indwelling arterial catheter 16 by a distance of less than 10 mm, and preferably between 3 to 10 mm, as such a distance may be sufficient to obtain a quality arterial blood sample due to the reasons provided above. In other embodiments, the blood draw device 60 may be configured to extend the distal end 70 of the catheter tube 66 out beyond the distal tip 26 of the indwelling arterial catheter 16 by a distance of up to 20 cm, 30 cm, or further, according to other non-limiting embodiments.

As shown in FIGS. 2-5, the housing 62 of blood draw device 60 can be an elongate member having a proximal end 72 and a distal end 74 and defining an inner volume 76. In some embodiments, the housing 62 may be formed of a pair of housing portions 78a, 78b that are coupled together to define the inner volume 76. The housing 62 may include one or more features or surface finishes on an outer surface thereof that can be arranged to increase the ergonomics of the blood draw device 60, which in some instances can allow a user to manipulate the blood draw device 60 with one hand (i.e., single-handed use).

The coupling device 64 of blood draw device 60 is provided at the distal end 74 of the housing 62, with the coupling device 64 providing for reversible coupling of the blood draw device 60 to catheter assembly 12, such as via needleless access connector 46 as shown in FIG. 1. In some embodiments, the coupling device 64 is configured as a lock 80 that includes a blunted cannula 82 and locking arms 84 for coupling to the needleless access connector 46 of catheter assembly 12, with the blunted cannula 82 and locking arms 84 forming three points of contact therewith. However, those of skill will appreciate that any connection or coupling, for example a luer, can be used, so long as the distal end 70 of catheter tube 66 may pass through the coupling device 64 to catheter assembly 12.

The advancement member 68 of blood draw device 60 includes a first portion 86 and a second portion 88. The first portion 86 is movably disposed along an upper surface 90 of the housing 62 and the second portion 88 is movably disposed within the inner volume 76 of the housing 62. The arrangement of the advancement member 68 and the housing 62 is such that a connecting portion (not shown) of the advancement member 68 that joins the first and second portions 86, 88 is seated within a slot 92 formed in the upper surface 90 of the housing 62—the slot 92 generally extending between the proximal and distal ends 72, 74 of the housing 62. As the first and second portions 86, 88 are joined together, movement of the first portion 86 along the upper surface 90 of the housing 62 results in a corresponding movement of the second portion 88 within the inner volume 76.

As shown in FIGS. 2-5, the first portion 86 of the advancement member 68 may be configured as a tab or tab having a contact surface 94a engageable by a user and an underside 94bthat is in contact with the outer surface 90 of the housing 62. In such embodiments, the upper surface 90 of the housing 62 can include a track 96, for example, a set of ribs, ridges, bumps, grooves, and/or the like along which the underside 94b of tab or protrusion advances when the advancement member 68 is engaged by a user. In this manner, a user can engage the first portion 86 of the advancement member 68 and can move the advancement member 68 relative to the housing 62.

As shown in FIGS. 2-5, the second portion 88 includes an opening 98 extending therethrough that is configured to grip or retain a portion of the catheter tube 66. Due to a portion of the catheter tube 66 being retained within the opening 98 of second portion, 94, movement of the advancement member 68 relative to housing 62 causes a corresponding movement of the catheter tube 66 relative to the housing 62. In this manner, the distal end 70 of the catheter tube 66 can be selectively moved out of or back into the inner volume 76 of the housing 62 as desired, such as advancing the distal end 70 of the catheter tube 66 out of the housing 62 when the blood draw device 60 has been coupled to the catheter assembly 12 and collection of an arterial blood sample is to be performed.

As indicated above, in advancing the distal end 70 of the catheter tube 66 out of the housing 62 and into the catheter assembly 12, blood draw device 60 may be configured to extend the distal end 70 of the catheter tube 66 out beyond the distal tip 26 of the indwelling arterial catheter 16 by a distance of less than 10 mm, and preferably between 3 to 10 mm, as such a distance is sufficient to obtain a quality arterial blood sample due to the reasons provided above. Advancement of the catheter tube 66 by this distance allows for a reduction in the length of the catheter tube and housing 62 and/or a limitation of the movement of advancement member 68 relative to housing 62. In some embodiments, introducer 62 may be configured to have a length L_{Intro} of between 103-110 mm, in association with the need for the extension length of catheter tube 66 to be less than what is typically required for use of a blood draw device with peripheral IV catheters. In some embodiments, housing 62 may be configured to have a length L_{Intro} of between 122-129 mm. In other embodiments, a spacer 100 (shown in phantom in FIG. 2) may be provided within the inner volume 76 of housing 62 at a location proximal to advancement member 68 (i.e., at proximal end 72 of housing 62), with the spacer 100 functioning to limit the path of travel of the advancement member 68 in association with the need for the extension length of catheter tube 66 to be less than what is typically required for use of a blood draw device with peripheral IV catheters.

As further shown in FIGS. 2-5, blood collection device 60 includes a secondary catheter 102 provided at the proximal end 72 of the housing 62. The secondary catheter 102 has a proximal end portion 104 and a distal end portion 106 and defines a lumen 108. A portion of the secondary catheter 102 is disposed within and extends through an opening 110 formed in the proximal end 72 of housing 62. As such, the proximal end portion 104 is at least partially disposed outside of the housing 62 and the distal end portion 106 is at least partially disposed within the housing 62, with the distal end portion 106 coupled to the second portion 88 of the advancement member 68. In some embodiments, the secondary catheter 102 can have a larger diameter than the catheter tube 66, which can function to limit, reduce, and/or substantially prevent hemolysis of a volume of blood as the volume of blood flows through the catheter 66 and the secondary catheter 102, as explained in further detail below. In this manner, when the blood draw device 60 is coupled to a syringe, evacuated container, etc., the secondary catheter 102 establishes fluid communication between the reservoir, source, pump, etc. and the catheter tube 66, as explained further below.

According to embodiments of the disclosure, the proximal end portion 104 of the secondary catheter 102 is coupled to and/or otherwise includes a coupler 114 configured to mate with a collection device 116 that is useable with (or is considered part of) the blood draw device 60 to collect an arterial blood sample for subsequent analysis, such as blood gas analysis of the sample. According to some embodiments, the coupler 114 may be configured as a luer connection (i.e., a female luer connection) configured to mate with a corresponding luer connection (i.e., a male luer connection) of the collection device 116. The coupler 114 physically and fluidically couples the secondary catheter 102 to the collection device 116, which as described further here below, may be any suitable vacuum or non-vacuum assisted blood sample collection device 116, such as a sampling syringe, vacutainer, luer lock access device (LLAD), a point-of-care (POC) sampling device, integrated POC cartridge, blood culture collection system, or the like. Non-limiting examples of vacuum or non-vacuum assisted blood sample collection devices may include a Vacutainer® Luer-Lok™ access device or Accustat™ device by Becton Dickinson and Company. In some embodiments, a venting feature 118 may be provided that functions to vent the vacuum or non-vacuum assisted collection device 116 prior to blood collection. As shown in the illustrated embodiment, the venting feature 118 may be provided on coupler 114, but it is recognized that venting feature 118 could be provided anywhere along the sampling fluid path, although preferably near the proximal end portion 104 of the secondary catheter 102.

As shown in FIGS. 1-5, in accordance with one aspect of the disclosure, the collection device 116 that is connected to blood draw device 60 (via coupler 114) is provided as a syringe 116a. In some embodiments, the syringe 116a may be pre-attached with a plunger 120 of the syringe 116a in an advanced position. When the catheter tube 66 of blood draw device 60 is advanced through the indwelling catheter 16 and into the artery 28, the syringe plunger 120 may be retracted to pull an undiluted arterial blood sample into the syringe 116a. The syringe 116amay contain blood preservatives/stabilizers therein to help maintain a quality of the sample. In other embodiments, the syringe 116a may begin in a retracted position and have a selective venting feature (e.g., vent 118 on coupler 114) via which the syringe 116a may be vented after the catheter tube 66 has been moved to the second, advanced position. Upon any venting of the syringe 116aand a subsequent sample collection and stabilization, the syringe 116a may be detached from the blood draw device 60 and the sample may then be dispensed to a POC blood diagnostic test cartridge 122 or provided to another blood analyzer test instrument.

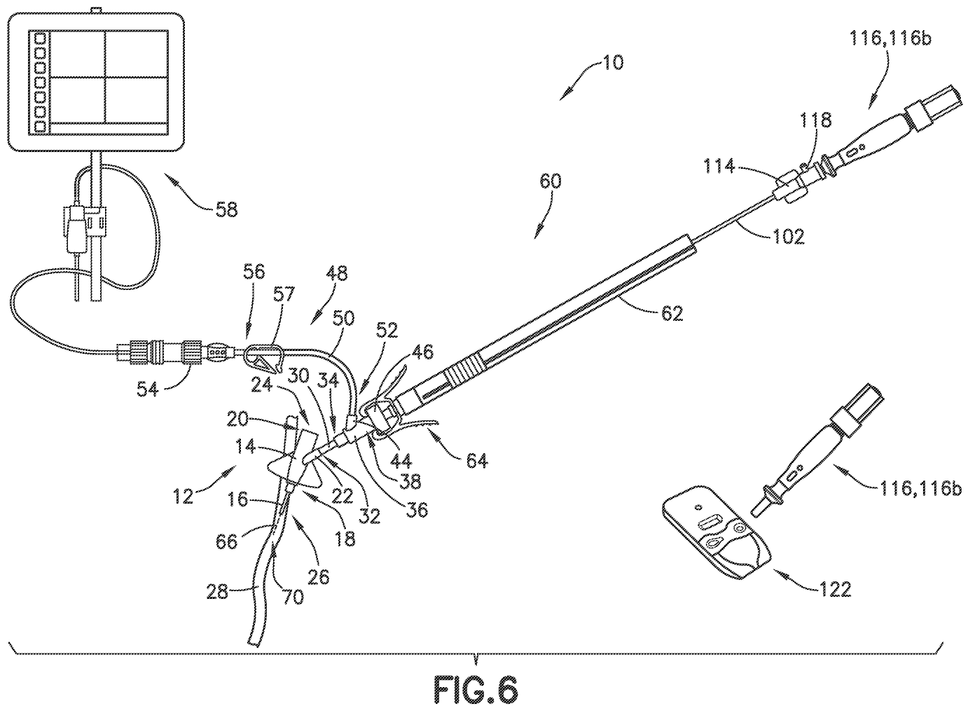

Referring now to FIG. 6, an arterial access system 10 is illustrated where the collection device 116 connected to blood draw device 60 (via coupler 114) is provided as a vacutainer collection device 116b, or a simplified/modified vacutainer that is vented, not requiring the vacuum tube to drive sampling volume collection. The vacutainer 116b may contain blood preservatives/stabilizers therein to help maintain a quality of the sample. Upon filling of the vacutainer 116b and blood stabilization, the vacutainer 116b may be detached from the blood draw device 60 and a blood sample can then be dispensed onto a POC diagnostic test cartridge 122 or provided to another blood analyzer test instrument.

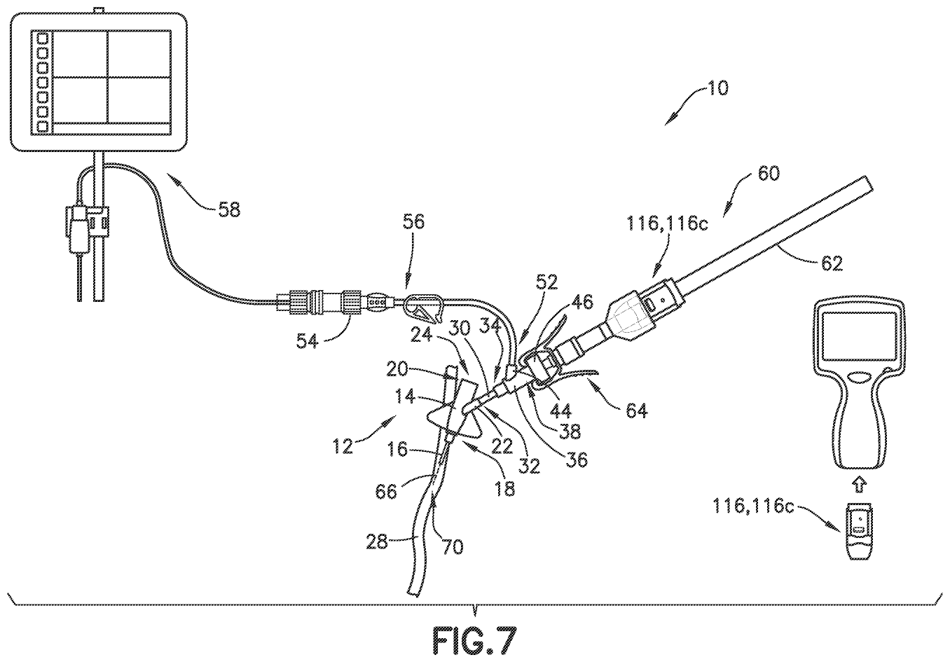

Referring now to FIG. 7, an arterial access system 10 is illustrated where the collection device 116 connected to blood draw device 60 is provided as an onboarded POC diagnostic cartridge 116c (e.g., an iStat test cartridge from Abbott Labratories). The diagnostic cartridge 116cmay be removably coupled to blood draw device 60 (i.e., coupled to housing 62) for direct collection of a micro-sample of arterial blood. With the diagnostic cartridge 116c, the system requires selective venting of the cartridge micro-fluidic fluid path to prevent filling until the distal tip 70 of the catheter tube 66 is directly in the artery 28 for undiluted blood sampling. Upon filling of the diagnostic cartridge 116c, it can be removed from the blood draw device 60 and inserted into a POC blood test instrument and analyzer.

As indicated above, the use of blood draw device 60 and collection device 116 with catheter assembly 12 enables a direct collection of an arterial blood sample. In accordance with aspects of the disclosure, the arterial blood draw can be performed either by connecting a vacuum-assisted blood sample collection device 116 (such as an evacuated syringe or LLAD with a vacutainer connected thereto), or by a non-vacuum-assisted blood sample collection device 116 (such as a vented syringe or other vented chamber). When using a non-vacuum assisted blood sample collection device 116 with the blood draw device 60, the arterial pressure acts to fill the vented collection device chamber, with the blood flow driven by the constant arterial pressure. In the case of a vacuum-assisted blood draw, a pressure gradient across a fluid path of the blood draw device (i.e., across catheter tube 66 and secondary catheter 102) is much higher, and thus shear driven hemolysis may affect the collection of the arterial blood sample.

Regarding the effect of shear driven hemolysis, max shear stress in a tubular fluid pathway (such as catheter tube 66 and secondary catheter 102) is determined by the flow rate and the smallest hydraulic diameter of the tube. Fluid flow in a tubular fluid pathway therethrough can be analyzed using Poiseuille's equation:

$$Q = \frac{π {D}^{4} Δ P}{128 μ L} = \frac{Δ P}{R f}$$

where ΔP is a change in pressure gradient across the length of the fluid pathway, D and L are the inner diameter and length, respectively, of the fluid pathway, μ is the viscosity of a fluid, and R_{f}=128 μL/πD⁴ is the fluid resistance. Since u is the viscosity of the fluid and not part of the extension tube geometry, a geometric factor Gf is defined such that R_{f} (the fluid resistance) is R_{f}=(128 μL/π)*G_{f}, where G_{f}=L/D⁴.

In some embodiments, the optimized fluid pathway may have multiple sections with lengths (L1, L2, L3, . . . ) and inner diameters of (D1, D2, D3, . . . ), the geometric factor is then:

$${G}_{f} = \frac{L 1}{D {1}^{4}} + \frac{L 2}{D {2}^{4}} + \frac{L 3}{D {3}^{4}}$$

In some embodiments, the optimized fluid pathway may have an inside diameter that changes over the length of the tube, the geometric factor is then:

$${G}_{f} = {\int }_{0}^{L} \frac{d l}{{D ( l )}^{4}}$$

In some embodiments, the optimized fluid pathway may have a cross section that has a non-circular inside diameter profile. The geometric factor can be determined by measuring the flow rate (Q) at given pressure (ΔP) with known viscosity (μ) fluid:

$${G}_{f} = \frac{πΔ P}{128 μ Q}$$

Since, in the case of arterial blood collection, the Gf value of the optimized fluid pathway would be different for a syringe draw or vented chamber and a vacutainer draw, the Gf value of the optimized fluid pathway can be the higher value between the syringe draw and vacutainer draw. Alternatively, the Gf value of the optimized fluid pathway can be the lower value between the syringe draw and vacutainer draw, and an accessory in the form of an extension set or connector can be connected to the Luer in the case where a higher Gf value is needed. In another alternative embodiment, the proximal end of the extension tube (i.e., secondary catheter 102) can consist of two ports where each port provides different Gf value.

In the case of a syringe draw or vented chamber, the blood draw occurs at constant flow rate due to constant driving arterial pressure. The max shear stress is constant where:

$${τ}_{max} = \frac{Δ P}{4 {G}_{f} D {min}^{3}}$$

where Dₘᵢₙ is the minimum diameter of the fluid pathway defined by catheter tube 66 and secondary catheter 102.

Based on the above, the G_{f} value of the optimized fluid pathway (i.e., catheter tube 66 and secondary catheter 102) may be selected depending on the blood draw methodology. In the case of a vacuum-assisted vacutainer draw, the G_{f} value of the optimized fluid pathway may be selected to reduce the max shear stress to be the same or less than the max shear stress of a typical vacuum-assisted, push button blood collection set—with a G_{f} value of equal to or greater than 220/Dₘᵢₙ³ being desirable, in accordance with aspects of the disclosure. In the case of a syringe draw or other vented chamber (i.e., a non-vacuum assisted device), the G_{f} value of the optimized fluid pathway may again be selected to reduce the max shear stress to be the same or less than the average max shear stress of a typical vacuum-assisted, push button blood collection set-with a G_{f} value of equal to or greater than 35/Dₘᵢₙ³ being desirable, in accordance with aspects of the disclosure.

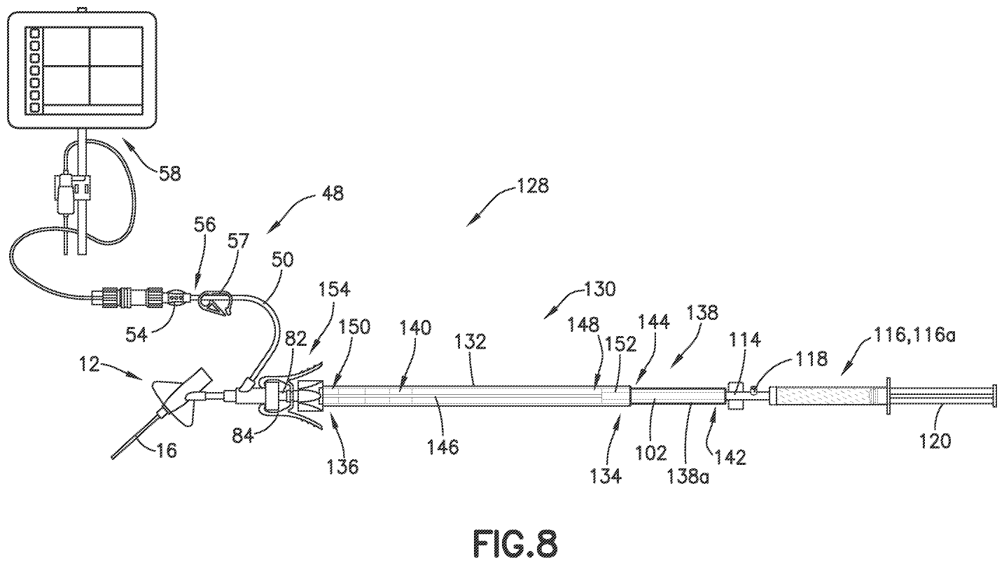

It is recognized that aspects of the disclosure are not limited to the specific blood draw device 60 shown and described in FIGS. 1-7, and that blood draw devices with other suitable constructions may also incorporate aspects of the disclosure. Referring now to FIG. 8, an arterial access system 128 is shown that includes a blood draw device 130 according to another embodiment of the disclosure. The blood draw device 130 includes a housing 132 having a proximal end 134 and a distal end 136, and an advancement member 138 slideably received within housing 132 (i.e., within an inner volume 140 of housing 132). In the illustrated embodiment, the advancement member 138 is provided as one or more telescopic cylinders 138a that are provided in a telescoping relationship with housing 132, such that advancement member 138 may be slideably received entirely, or almost entirely, within the inner volume 140 of housing 132. Advancement member 138 also includes a proximal end 142 and a distal end 144 and, in non-limiting embodiments, advancement member 138 may have a variable diameter along its length. As one example, the distal end 144 of advancement member 138 may have a larger diameter than other portions of advancement member 138 such that, as advancement member 138 is retracted, one or more features on housing 132 may interact with the enlarged portion of advancement member 138 to prevent pulling advancement member 138 completely out of housing 132. As another example, the distal end 144 of advancement member 138 may have a smaller diameter than other portions of advancement member 138, to keep the advancement member 138 in position at a blood draw forward condition, so that a hand of the operator is freed up to manipulate additional components (e.g., a vacutainer tube).

Blood draw device 130 further includes a catheter tube 146 having a proximal end 148 and a distal end 150. Catheter tube 146 is received within the inner volume 140 of housing 132, and may be advanced and/or retracted relative to housing 132 by displacement of the advancement member 138 relative to the housing 132. In some embodiments, the catheter tube 146 may be joined to advancement member 138 via a fitting 152 provided at the distal end 144 of advancement member 138, such that displacement of the advancement member 138 relative to the housing 132 causes a corresponding displacement of catheter tube 146. In non-limiting embodiments, catheter tube 146 may be advanced from a first positon in which distal end 150 of catheter tube 146 is within housing 132, to a second position in which a distal end 150 of catheter tube 146 is positioned distally of housing 132 (and also positioned distally of catheter 16), as previously described regarding blood draw device 60 and operation thereof.

Blood draw device also includes a coupling device 154 thereon which may be identical to the coupling device shown and described in the blood draw device of FIGS. 1-7. That is, coupling device 154 is configured as a lock 80 that includes a blunted cannula 82 and locking arms 84 for coupling to the needleless access connector 46 of catheter assembly 12, with the blunted cannula 82 and locking arms 84 forming three points of contact therewith. However, it is appreciated that alternative embodiments of blood draw device 130 may include a coupling device 154 of another type to secure blood draw device 130 to catheter assembly 12, including luer connections, clips, blunt plastic cannulae, blunt metal cannulae, hybrid luers (e.g., with a cannula) friction fits, and the like.

According to aspects of the disclosure, a secondary catheter 102 may be routed through the telescopic cylinder 138a of advancement member 138, with the secondary catheter 102 providing a fluid connection between the catheter tube 146 and a coupler 114 provided at the proximal end portion 104 of the secondary catheter 102-with the coupler 114 configured to mate with a collection device 116. Although shown in FIG. 8 as a syringe 116a, the collection device 116 may be configured as any of the collection devices 116 shown and described in FIGS. 1-7, with the collection device 116 attached to blood draw device 130 to provide an arterial access system for facilitating improved arterial line blood collection for arterial blood gas sampling. That is, the blood draw device 130 may accommodate use of any of a number of different vacuum-assisted or non-vacuum-assisted blood collection devices 116 therewith for collection of the blood sample, with the blood draw device configurable to prevent hemolysis during the sample collection, such as by controlling the sizing of the device, including the geometric factor G_{f} of the device (i.e., of the fluid pathway therein).

Beneficially, embodiments of the disclosure thus provide an arterial access system for facilitating improved arterial line blood collection for arterial blood gas sampling and continuous pressure monitoring. The arterial access system includes a catheter assembly having a blood draw device attached thereto that may be used to directly collect an arterial blood sample from the patient via the indwelling arterial catheter, with such blood sample being used for blood gas analysis. The arterial access system simultaneously provides for continuous blood pressure monitoring via use of an extension set attachable to the catheter assembly and that provides arterial blood to hemodynamic monitoring system connected thereto. The blood draw device may accommodate use of any of a number of different vacuum-assisted or non-vacuum-assisted blood collection devices therewith for collection of the blood sample, with the blood draw device configurable to prevent hemolysis during the sample collection.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is

1. A blood draw device useable with an indwelling arterial catheter for performing arterial blood collection, the blood draw device comprising:
a catheter tube;
a housing having a proximal end portion and a distal end portion and defining an inner volume configured to movably receive the catheter tube;
an advancement member movably coupled to the housing and configured to move relative to the housing to move the catheter tube between a first position, in which the catheter tube is disposed within the housing, and a second position, in which a distal end of the catheter tube is disposed beyond the distal end portion of the housing and past a distal tip of the indwelling arterial catheter;
a secondary catheter coupled to the advancement member and extending out proximally therefrom and out through the proximal end portion of the housing, the secondary catheter in fluid communication with the catheter tube; and
a collection device coupled to the proximal end of the secondary catheter, so as to be in fluid communication with the secondary catheter and the catheter tube;
wherein a fluid path comprising the catheter tube and the secondary catheter has a controlled geometric factor, Gf, defined as a ratio of a length of the fluid path, L, to an inner diameter of the fluid path, D.

2. The blood draw device of claim 1, wherein the blood draw device comprises a coupler connected to a proximal end of the secondary catheter, and wherein the collection device is connected to the coupler.

3. The blood draw device of claim 2, wherein the collection device comprises a vacuum-assisted collection device.

4. The blood draw device of claim 2, wherein the collection device comprises a non-vacuum-assisted collection device.

5. The blood draw device of claim 4, wherein the coupler comprises a venting feature configured to selectively vent the non-vacuum-assisted collection device.

6. The blood draw device of claim 2, wherein the geometric factor, Gf, is equal to or greater than 35/Dₘᵢₙ³ for a non-vacuum-assisted collection device and between equal to or greater than 220/Dₘᵢₙ³ for a vacuum-assisted collection device.

7. The blood draw device of claim 6, wherein for the secondary catheter having a constant inner diameter, the geometric factor, Gf, is defined as:
$$G f = \frac{L}{{D}^{4}} .$$

8. The blood draw device of claim 6, wherein for the secondary catheter having have multiple sections with lengths L1, L2, L3 and inner diameters of D1, D2, D3, the geometric factor, Gf, is defined as:
$${G}_{f} = \frac{L 1}{D {1}^{4}} + \frac{L 2}{D {2}^{4}} + \frac{L 3}{D {3}^{4}} .$$

9. The blood draw device of claim 6, wherein for the secondary catheter having a changing inside diameter over the length thereof, the geometric factor, Gf, is defined as:
$${G}_{f} = {\int }_{0}^{L} \frac{d l}{{D ( l )}^{4}} .$$

10. The blood draw device of claim 1, wherein the housing and/or the advancement member are configured such that, when the catheter tube is moved to the second position, the distal end of the catheter tube extends out 10 mm or less past the distal tip of the indwelling arterial catheter.

11. The blood draw device of claim 1, wherein the housing and/or the advancement member are configured such that, when the catheter tube is moved to the second position, the distal end of the catheter tube extends out between 3-10 mm past the distal tip of the indwelling arterial catheter.

12. The blood draw device of claim 1, wherein the housing has a length of 103-110 mm or 122-129 mm.

13. The blood draw device of claim 1, comprising a spacer positioned within the inner volume of the housing and proximally from the advancement member, the spacer limiting movement of the advancement member along the housing.

14. A method of manufacturing an arterial blood draw device operable to collect an arterial blood sample, the method comprising:
providing a catheter tube;
housing the catheter tube within an inner volume of a housing having a proximal end portion and a distal end portion, the catheter tube movable within the housing;
coupling an advancement member to the catheter tube and to housing, with the advancement member movable relative to the housing, so as to cause a corresponding movement of the catheter tube between a first position, in which the catheter tube is disposed within the housing, and a second position, in which a distal end of the catheter tube is disposed beyond the distal end portion of the housing;
connecting a secondary catheter to the advancement member, the secondary catheter extending proximally from the advancement member and out through the proximal end portion of the housing, the secondary catheter in fluid communication with the catheter tube;
connecting a collection device at a proximal end of the secondary catheter, the collection device comprising one of a vacuum-assisted collection device and a non-vacuum-assisted collection device; and
configuring the catheter tube and the secondary catheter to collectively provide a fluid path having a geometric factor, Gf, defined as a ratio of a length of the fluid path, L, to an inner diameter of the fluid path, D, with the geometric factor, Gf, having a first value or value range when the collection device is a vacuum-assisted collection device and having a second value or value range when the collection device is a non-vacuum-assisted collection device.

15. The method of claim 14, wherein the geometric factor, Gf, is equal to or greater than 35/Dₘᵢₙ³ for a non-vacuum-assisted collection device and between equal to or greater than 220/Dₘᵢₙ³ for a vacuum-assisted collection device.

16. The method of claim 14, wherein the housing and/or the advancement member are configured such that, when the catheter tube is moved to the second position, the distal end of the catheter tube extends out 10 mm or less past the distal tip of the indwelling arterial catheter.

17. The method of claim 14, wherein for the secondary catheter having a constant inner diameter, the geometric factor, Gf, is defined as:
$$G f = \frac{L}{{D}^{4}} .$$

18. The method of claim 14, wherein for the secondary catheter having have multiple sections with lengths L1, L2, L3 and inner diameters of D1, D2, D3, the geometric factor, Gf, is defined as:
$${G}_{f} = \frac{L 1}{D {1}^{4}} + \frac{L 2}{D {2}^{4}} + \frac{L 3}{D {3}^{4}} .$$

19. The method of claim 14, wherein for the secondary catheter a changing inside diameter over the length thereof, the geometric factor, Gf, is defined as:
$${G}_{f} = {\int }_{0}^{L} \frac{d l}{{D ( l )}^{4}} .$$

20. The method of claim 14, further comprising constructing the housing to have a length of 103-110 mm or 122-129 mm.

21. The method of claim 20, comprising constructing the housing and/or coupling the advancement member to the housing such that, when the catheter tube is moved to the second position, the distal end of the catheter tube extends out 10 mm or less past the distal end portion of the housing.
